(19) United States
(12) Patent Application Publication
(10) Pub. No.: US 2013/0261380 A1
(43) Pub. Date: Oct. 3, 2013

(54) Devices and Methods for the Treatment of Obesity

(71) Applicants: Kim Otto, Redwood City, CA (US); Pankaj Rathi, Mountain View, CA (US); Zachary Warden-Gabaldon, Palo Alto, CA (US)

(72) Inventors: Kim Otto, Redwood City, CA (US); Pankaj Rathi, Mountain View, CA (US); Zachary Warden-Gabaldon, Palo Alto, CA (US)

(73) Assignee: VIBRYNT, INC., Redwood City, CA (US)

(21) Appl. No.: 13/437,632

(22) Filed: Apr. 2, 2012

(51) Int. Cl. A61F 2/00

(52) U.S. Cl. CPC A61F 2/0063
USPC 600/37

(57) ABSTRACT

A device includes a body having at least first and second edges and a plurality of laces configured and dimensioned to be threaded across the stomach. Multiple devices can be positioned about a stomach. Methods for implanting the device and for treating a patient are provided.

### BACKGROUND OF THE INVENTION

Embodiments of the present invention relate generally to medical devices and methods and more particularly to minimally invasive devices, systems and methods for treating obesity.

Obesity has become a major health concern, both nationally and internationally. The National Center for Health Statistics (NCHS) estimates that over 120 million Americans are overweight, including about 56% of the adult population. Of these, about 52 million are considered obese, as measured by a body mass index (BMI) of 30% or greater. In Europe, an estimated 77 million people are obese, as measured by the same standard. This problem is not limited to western nations, as many developing countries are reported to have obesity rates over 75% of the adult population.

Co-morbidities that are associated with obesity include, but are not limited to type II Diabetes, high blood pressure, sleep apnea, stroke and arthritis, the symptoms of which often tend to be lessened or alleviated upon loss of weight by a person so affected.

In the U.S., options for treatment of obesity are currently quite limited. Current treatment methodologies typically rely upon surgically introducing a “malabsorptive” environment in the gastro-intestinal tract, a restrictive environment, or a combination of these. One available treatment method is gastric bypass surgery and another is referred to as gastric banding (one of these techniques if referred to as the LAPBAND™ procedure). These procedures are limited to only those patients with a BMI over 40 (or over 35, with co-morbidities present).

Gastric bypass procedures incur a great deal of morbidity and create a malabsorptive state in the patient by bypassing a large portion of the intestines. Serious side effects, such as liver failure have been associated with this procedure, as well as chronic diarrhea. Another surgical procedure that has a high degree of morbidity associated with it is known as the “Gastric Bypass Roux-en-Y” procedure. This procedure reduces the capacity of the stomach by creating a smaller stomach pouch. The small space holds only about fluid ounce. A tiny stomach outlet is also surgically created to slow the speed at which food leaves the stomach. Staples are used to create a small (15 to 20 cc) stomach pouch, with the rest of the stomach being stapled completely shut and divided from the stomach pouch. The small intestine is divided just beyond the duodenum, brought up, and connected to the newly formed stomach pouch. In addition to the considerable morbidity associated with this procedure, other disadvantages include “dumping syndrome”, where stomach contents are literally “dumped” rapidly into the small intestine which may lead to nausea, weakness, sweating, faintness, and diarrhea; hernias resulting from the surgery; gallstones; leakage of the connection between the pouch and the intestine; stretching of the pouch that was formed; nutritional deficiencies; and possible dehiscence of the staples.

The LAPBAND™ is a band that, when placed, encircles the fundus-cardia junction and is inflatable to constrict the same. It does not reduce the volume of the stomach, but rather restricts passage of food into the stomach, the theory being that the patient will feel satiety with a much less volume of food than previously. Although the LAPBAND™ procedure is less invasive than a gastric bypass procedure, it also typically achieves less weight loss. Further, it is not a simple procedure and requires a substantial amount of training by a surgeon to become proficient in performing the procedure. Also, a substantial amount of dissecting and suturing is required because the pathway by which the band is introduced is not an existing pathway, and must be established by dissection. Great care is required to avoid blood vessels and nerves that may be in the intended pathway to be created by the dissection. After placing the band around the fundus-cardia junction, the ends of the band must be connected together and then it must be cinched down into place. Additionally, complications such as erosion at the fundus-cardia junction, slippage of the band from its intended location, nausea/vomiting, gastroesophageal reflux, dysphagia and lack of effectiveness in causing weight loss have been reported.

Intra-gastric balloons have also been placed, in an attempt to fill a portion of the volume in the stomach, with the theory being that it will then require less food than previously, to give the patient a sensation of fullness or satiety. This procedure involves delivery of a balloon (typically, trans-orally) to the interior of the stomach and inflation of the balloon to take up a portion of the volume inside the stomach. However, intra-gastric balloons may also lead to complications such as obstruction, vomiting and/or mucosal erosion of the inner lining of the stomach. The balloon can break down over extended exposure to the stomach's acids, and in some cases, after breaking down, the balloon translated through the intestines and caused a bowel obstruction.

Gastrointestinal sleeves have been implanted to line the stomach and/or a portion of the small intestines to reduce the absorptive capabilities of the small intestine and/or to reduce the volume in the stomach, by reducing the available volume to the tubular structure of the graft running therethrough. Although weight loss may be effective while these types of devices are properly functioning, there are complications with anchoring the device within the stomach/GI tract, as the stomach and GI tract function to break down things that enter into them and to move/transport them through. Accordingly, the integrity of the anchoring of the device, as well as the device itself may be compromised over time by the acids and actions of the stomach and GI tract.

A sleeve gastrectomy is an operation in which the left side of the stomach is surgically removed. This results in a much reduced stomach which is substantially tubular and may take on the shape of a banana. This procedure is associated with a high degree of morbidity, as a large portion of the stomach is surgically removed. Additionally, there are risks of complications such as dehiscence of the staple line where the staples are installed to close the surgical incisions where the portion of the stomach was removed. Further, the procedure is not reversible.

In the laparoscopic duodenal switch, the size of the stomach is reduced in similar manner to that performed in a sleeve gastrectomy. Additionally, approximately half of the small intestine is bypassed and the stomach is reconnected to the shortened small intestine. This procedure suffers from the same complications as the sleeve gastrectomy, and even greater morbidity is associated with this procedure due to the additional intestinal bypass that needs to be performed. Still further, complications associated with malabsorption may also present themselves.

An inflatable gastric device is disclosed in U.S. Pat. No. 4,246,893, in which a balloon is inserted anteriorly of the stomach and posteriorly of the left lobe of the liver. The balloon is then inflated to compress the stomach so that it fills with less food that would ordinarily be possible. Not only does this device compress the stomach, but it also compresses the liver, as seen in FIG. 5 of the patent, which may cause complications with the liver function. Additionally, the balloon is simply placed into this location, and there is no assurance that it will not migrate and lose its effectiveness in compressing the stomach to the degree intended. Still further, the balloon is of a simple spherical design, and, as such, extends pressure outwardly in all directions, 360 degrees in all planes. Accordingly, the liver is compressed just as much as the stomach is. Also, the compression forces against the stomach are not ideal, as the spherical balloon conformation does not match the conformation of the expanding stomach. The stomach is not spherical when expanded, or concave with a constant radius of curvature, but expands into a designated space that allows the fundus to expand preferentially more than other parts of the stomach.

Brazzini et al. in WO2005/18417 discloses at least two or more expandable devices used to treat obesity, in which the devices are inserted through the abdominal wall and anchored subcutaneously or to the stomach wall to exert pressure against the external surface of the stomach wall.

U.S. Patent Publication No. 2005/0261712 to Balbierz et al. describes capturing a device against the outer surface of the stomach wall to form a restriction that appears to function similarly to the restriction imposed by the LAPBAND™. The anchoring of the devices disclosed relies upon placement of features against the internal wall of the stomach to form an interlock with the device which is placed against the external wall of the stomach.

U.S. Patent Publication No. 2005/0267533 to Gertner discloses devices for treatment of obesity that use one or more anchoring mechanisms that are passed through the wall of the stomach to establish an anchor. The stomach is reduced in size by passing the devices through the stomach wall on opposite sides of the stomach and compressing the walls together to eliminate a portion of the interior space within the stomach. Gertner also discloses an embodiment in which an extra-gastric balloon is placed anteriorly of the stomach and attached to the abdominal wall using one of the anchoring mechanisms described.

U.S. Pat. No. 6,981,978 to Gannoe discloses devices for reducing the internal cavity of the stomach to a much smaller volume, which may be used to carry out a bypass procedure. Stapling is employed to isolate the smaller volume in the stomach, and thus the same potential disadvantages are present as with other stapling procedures described herein.

U.S. Pat. No. 6,186,149 to Pacella et al. describes an occluder device that can be used as a dietary control device (see FIG. 8C). The occluder device is placed against the wall of the stomach and inflated to press inwardly on the stomach wall. A frame is wrapped around the stomach wall and is inflated to press against the stomach wall. However, there is no disclosure of how the frame might be adjusted to maintain a position relative to the stomach wall as the size of the stomach varies.

Gastric reduction techniques have been attempted, such as by inserting instruments trans-orally and reducing the volume of the stomach by stapling portions of it together. However, this technique is prone to failure due to the staples pulling through the tissues (i.e., dehiscence) that they are meant to bind.

Techniques referred to as gastric pacing endeavor to use electrical stimulation to simulate the normal feedback mechanisms of a patient that signal the brain that the patient is full, or satiated. While these techniques are less invasive than some of the other existing treatments, statistics to date have shown that the amount of weight lost by using such techniques is less than satisfactory.

Currently marketed drugs for weight loss, such as XENICAL®, MERIDIA® and Fen Phen have largely failed, due to unacceptable side effects and complications, and sometimes to an ineffective amount of weight loss. Other drugs that are on the horizon include ACCOMPLIA® and SYMLIN®, but these are, as yet, unproven.

Methods that places devices such as bands, balloons, or wraps on the extra-gastric surface of the stomach carry the risk of creating erosions on the stomach surface. Such erosions can lead to migration of the device, even through to the stomach. Erosions carry the risk of spillage of stomach content into the abdominal cavity, which can be a life-threatening event. Erosions can be caused by sharp edges or focused pressure on the stomach wall. Constriction of the stomach applies force to the stomach wall and devices that do so can create erosions.

Some procedures create folds or plications in the stomach wall to reduce the stomach volume. These methods often create the plication using an invasive mechanical device such as a transgastric anchor (see, e.g., U.S. Patent Publication 2008/0255592 to Hsu et al.). A device placed transgastrically may have its interior section dissolved by the stomach thereby creating an aperture for spillage of stomach content into the abdominal cavity.

Regardless of the means of creating a plication, plication carries the risk of necrosis of the stomach after plication possibly due to the blood supply being cut off from compression of blood vessels. More generally, many of the methods of constricting the stomach place unacceptable burdens on the major and minor blood vessels around the stomach, sometimes compressing them and sometimes dissecting them.

The risk and invasiveness factors of currently available surgeries are often too great for a patient to accept to undergo surgical treatment for his/her obesity. Accordingly, there is a need for less invasive, yet effective surgical treatment procedures for morbidly obese patients (patients having a BMI of 35 or greater). Also, since the current surgical procedures are currently indicated only for those patients having a BMI of 40 or greater, or 35 or greater when co-morbidities are present, it would be desirable to provide a surgical procedure that would be available for slightly less obese patients, e.g., patients having a BMI of 30 to 35 who are not indicated for the currently available surgical procedures, as well as for more obese patients.

There is a need for devices and methods for treating obesity with minimally invasive devices that substantially preserve the physiological integrity of the blood vessels around the stomach while providing an adjustable, constrictive force on the stomach.

### BRIEF SUMMARY OF THE INVENTION

Certain embodiments of the present invention are related to a method for the treatment of obesity. The method includes accessing an extra-gastric abdominal space of a patient and introducing a constricting device into the extra-gastric abdominal space. The constricting device includes a body with opposed first and second edges and a first plurality of laces with opposed ends. The method further includes positioning the body about the stomach of the patient and threading at least some of the first plurality of laces across the inner curvature of the stomach wherein the laces are threaded so as to avoid compression of blood vessels present near the inner curvature of the stomach. The method further includes fastening at least some of the first plurality of laces to the second edge of the body, tightening at least some of the first plurality of laces to constrict the stomach, and securing the tightened laces.

Certain embodiments of the present invention are related to a method for the treatment of obesity. The method includes creating a fold in the exterior surface of the stomach so that at least two different areas of serosa of the exterior surface of stomach become in contact. The fold is maintained through the use of devices of certain embodiments of the invention.

Certain embodiments of the present invention are related to a device for the treatment of obesity. The device includes a body having first and second edges wherein the body is configured and dimensioned to be positioned about the stomach of a patient and a plurality of laces wherein the laces are configured and dimensioned to be threaded across the inner curvature of the stomach.

### BRIEF DESCRIPTION OF SEVERAL VIEWS OF THE DRAWINGS

FIG. 1 illustrates the stomach and several of the blood vessels present near the stomach.

FIG. 2A is a cross-sectional illustration of a device and method according to certain embodiments of the invention in which the device is constricting the stomach.

FIG. 2B is a cross-sectional illustration of a device and method according to certain embodiments of the invention in which the constricting device is facilitating the formation of a plication in the stomach.

FIGS. 3A, 3B, and 3C are each illustrations of different embodiments of the device according to certain embodiments of the invention.

FIG. 4 is an illustration of a device body according to certain embodiments of the invention.

FIG. 5A is a plan view illustration of a lace according to certain embodiments of the invention.

FIG. 5B is a side view illustration of a lace according to certain embodiments of the invention.

FIG. 6 is a perspective illustration of a device according to certain embodiments of the invention in which the device includes selectively inflatable bladders.

FIG. 7 is an illustration of an inflation device according to certain embodiments of the invention.

FIG. 8 is an illustration of a device according to certain embodiments of the invention in which one device body is placed about the anterior of the stomach and another device body (not shown) is placed about the posterior of the stomach.

### DETAILED DESCRIPTION OF THE INVENTION

Embodiments of the present invention are useful in the surgical treatment of obesity. The description, figures, and examples herein relate to devices and methods for the containment or constriction of the stomach.

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

Short summaries of certain terms are presented in the description of the invention. Each term is further explained and exemplified throughout the description, figures, and examples. Any interpretation of the terms in this description should take into account the full description, figures, and examples presented herein.

The singular terms “a,” “an,” and “the” include plural referents unless the context clearly dictates otherwise. Thus, for example, reference to an object can include multiple objects unless the context clearly dictates otherwise. Similarly, references to multiple objects can include a single object unless the context clearly dictates otherwise.

The terms “substantially,” “substantial,” and the like refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

The terms “fasten,” “fastening,” “fastened,” “fastener” and the like refer to one part being coupled to another part in a relatively fixed manner which allows for some movement of one or both parts. The two parts may be fastened directly or through one or more intermediate parts provided that the intermediate parts preserve the relatively fixed manner of the coupling. As a non-limiting example, a shoelace is fastened to a shoe upper by passing the shoelace through the grommets on the shoe upper.

The terms “attach,” “attaching,” “attached,” “attachment” and the like refer to one part being coupled to another part in a substantially fixed manner. The two parts may be attached directly or through one or more intermediate parts provided that the intermediate parts preserve the substantially fixed manner of the coupling.

The terms “secure,” “securing,” “secured,” “securing,” and the like refer to one part being coupled to another part in a firmly fixed manner which prevents the movement of one part relative to the other.

An “edge” or “edges” refer to the margins of a part and include the extreme margin as well as the area at or near the extreme margin. Edges are not limited to linear shapes.

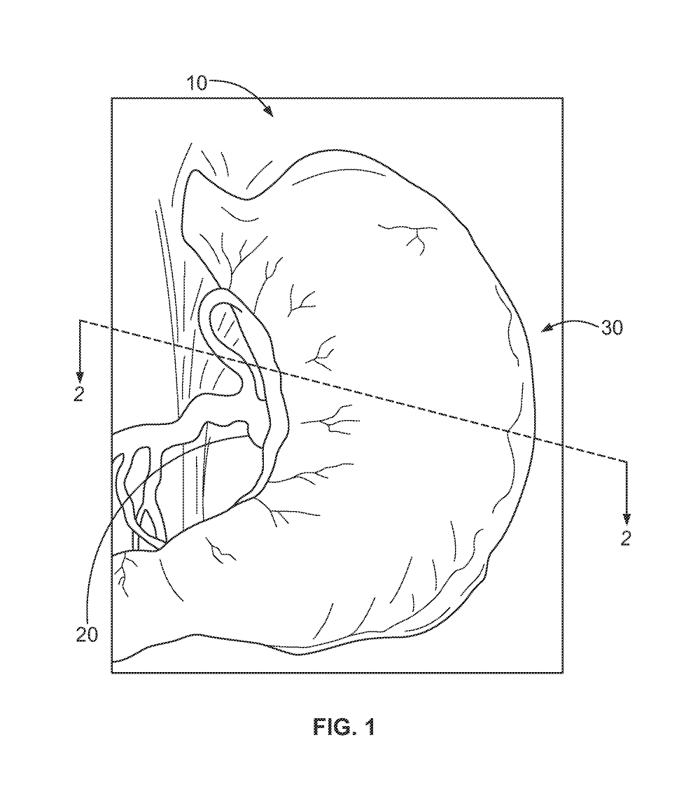

FIG. 1 illustrates the stomach 10 and several of the blood vessels present near the stomach. Some of these blood vessels supply blood to the stomach itself and other blood vessels supply blood to internal organs such as the spleen or the liver. For example, located along or nearby the inner curvature 20 (or lesser curvature) of the stomach are the left gastric artery, the celiac trunk, the common hepatic artery, the aorta, and the right gastric artery. Other smaller branch arteries emanate from some of these arteries, making the inner curvature 20 an area rich with blood vessels important for the function of abdominal organs. Similarly, located along or nearby the outer curvature 30 (or greater curvature) of the stomach are the right and left gastroepoploic arteries, sections of the splenic artery, and a series of short gastric arteries. As compared to inner curvature 20, outer curvature 30 has a lower density of blood vessels, both those that supply the stomach 10 and those that supply other organs or muscles. In abdominal surgeries that involve the stomach, the area around the outer curvature tends to tolerate surgical dissection more readily than the area around the inner curvature in part because of the lower density of blood vessels in the area around the outer curvature.

One of the novel aspects of the present invention is an appreciation of the importance of substantially maintaining the integrity of the blood vessels on both the inner curvature and the outer curvature of the stomach while still providing an effective treatment for obesity. Unlike devices and methods of the prior art that often depict and treat the stomach as a freestanding organ with little to no regard for the important blood vessels attached to or nearby it, certain embodiments of the present invention are configured to conform with the actual anatomy of the stomach and its milieu. Unlike devices and methods of the prior art, certain embodiments of the present invention are configured to facilitate the preservation of many of these blood vessels both during the initial implantation of the device and during the lifetime of the device.

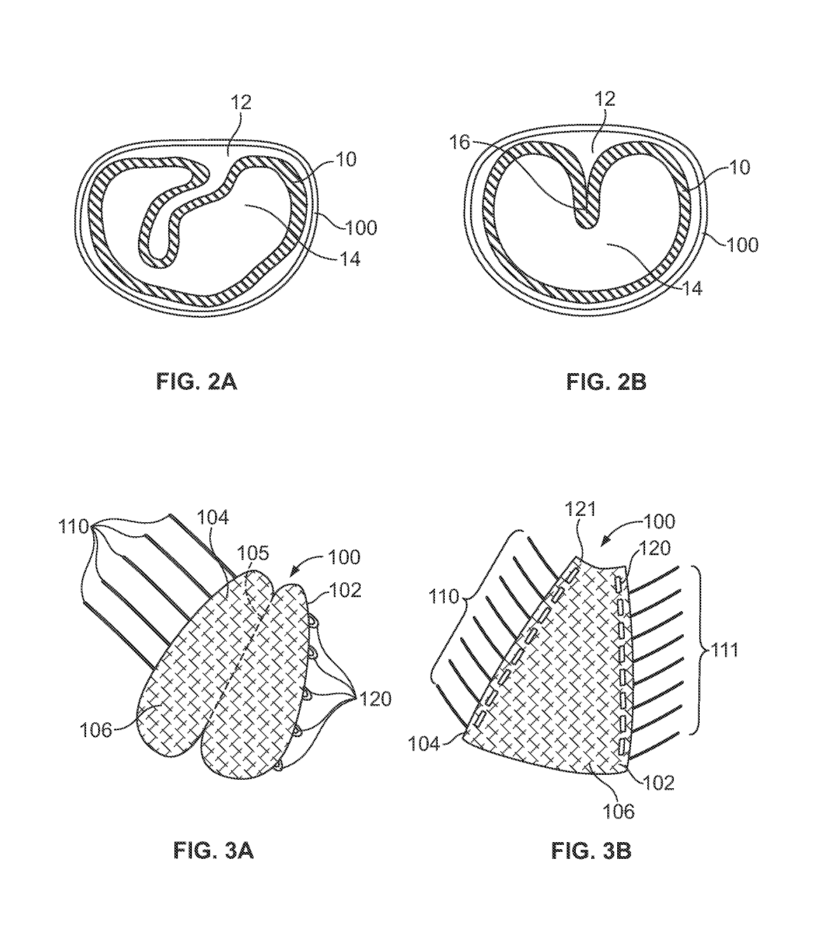

FIG. 2A is a cross-sectional illustration of a device and method according to certain embodiments of the invention in which the device is constricting the stomach.

FIG. 2B is a cross-sectional illustration of a device and method according to certain embodiments of the invention in which the constricting device is facilitating the formation of a plication in the stomach. FIGS. 2A and 2B schematically depict the approximate, gross cross-section of stomach 10 and device 100 at line 2 from FIG. 1 after device 100 has been applied to the stomach according to certain embodiments of the invention that are described in more detail in later sections of this disclosure. In FIGS. 2A and 2B, the device 100 is illustrated simplistically to more easily depict the plication method of certain embodiments of the invention and it is understood that the various embodiments of the device described and depicted herein can be employed in the method depicted in these figures.

Referring to FIGS. 2A and 2B, stomach 10 has an outer surface 12 and an inner surface 14. Outer surface 12 is composed of serosa, also knows as serous membrane. Serosa is composed of a secretory epithelial layer and a connective tissue layer underneath. Generally speaking, when one area of serosa is placed in contact with another area of serosa, the area may grow together to form a serosa-to-serosa joint. FIG. 2A depicts a method according to certain embodiments of the invention of constricting stomach 10 using device 100 to first form a fold in stomach 10 that brings areas of outer surface 12 together. The user may encourage the formation of the fold manually. FIG. 2B depicts a method according to certain embodiments of the invention of further constricting stomach 10 to bring area of outer surface 12 into contact such that a serosa-to-serosa joint may be formed. Regardless of whether a serosa-to-serosa joint occurs physiologically, FIG. 2B illustrates a method according to certain embodiments of the invention of constricting stomach 10 such that plication 16 is formed in stomach 10. Plication 16 reduces the volume of stomach 10 and, independently or in combination with the constricting action of device 100 on stomach 10, facilitates the treatment of obesity.

It is understood that within the scope of certain embodiments of the present invention additional means may be used to promote the formation of plication 16 and the serosa-to-serosa joint. Meshes or other similar materials that promote tissue ingrowth maybe be placed within the fold of stomach 10 during the constricting process. Tissue ingrowth promoters that are known in the art or may be subsequently developed can be used within the scope of certain embodiments of the invention. Additionally, the use of chemical or biological agents known or subsequently developed to promote serosa-to-serosa joints or stable plications are also within the scope of certain embodiments of the present invention. However, it is also understood that these other means are optional and that one of the advantages of certain embodiment of the invention is that a stable plication and/or a serosa-to-serosa joint can be formed without such additional means.

A further advantage of certain devices and methods of the present invention as described in detail herein is that plication 16 can be formed without the blood supply to the stomach being cut off due to compression or dissection of blood vessels. As compared to some prior art instances of stomach plication, the design, configuration, and execution of devices and methods of certain embodiments of the invention substantially avoid compressing or dissecting major blood vessels by allowing parts of the device to be placed away from the blood vessels yet also to apply and to maintain the necessary constrictive and/or plication-promoting conditions.

Another advantage of certain devices and methods of the present invention as described in detail herein is the relatively wide distribution of constrictive forces about stomach 10. FIGS. 2A and 2B grossly depict device 100 as circumscribing stomach 10. Although devices and methods that do not completely circumscribe or enclose the stomach are within the scope of certain embodiments of the invention, it is understood that one aspect of certain embodiments of the invention a relatively high level of surface contact of such embodiments of the device with the stomach. Spreading a force over a relatively larger surface area decreases the amount of force that any given point within that surface area experiences. Without being bound by any particular theory or mode of action, when this concept is applied to the stomach through devices and methods of the invention the constricted stomach is less likely to experience erosions.

Yet another advantage of certain devices and methods of the present invention as described in detail herein is the use of relatively smooth contact points where device 100 interacts with stomach 10. Certain devices of the prior art have relatively sharp or abrupt edges or device features that can act as focal points for forces on the stomach. Such focal points can lead to erosions. The various parts of certain embodiments of the invention are designed and configured to present rounded or “soft” edges and features to the stomach and the blood vessels in the area of the stomach so as to reduce the incidence of erosions and the compression or dissection of blood vessels.

Devices placed about the stomach according to certain embodiments of the invention are made with a device body having suitable dimensions and configurations. In certain embodiments of the invention, the device body is made from a flexible material and/or stretchable material. Some example of types of materials suitable for making a device body include biocompatible fabrics, cloths, and textiles made through weaving, knitting, spreading, crocheting, bonding or other techniques, such as silicone, polyester, polyvinyl chloride, polyethylene, poly ether-ether ketone, polycarbonate, polyurethane, polypropylene, and nylon. Other examples of types of materials suitable for making a device body include biocompatible sheets, films, or meshes made by conventional techniques; such sheets, films, or meshes can be made from biocompatible polymers such as silicone, polyester, polyvinyl chloride, polyethylene, poly ether-ether ketone, polycarbonate, polyurethane, polypropylene, and nylon. Device bodies may also be made from other plastics or metals using fabrication methods and techniques that generate sufficient strength, flexibility, and rigidity, among other properties, to make suitable device bodies as described herein.

The device body according to certain embodiments of the invention can range in thickness from about 0.05 mm to about 20 mm. To obtain such thicknesses, the fabrics and films useful for making the device body may be used as a single layer or in multiple layers. Further, the fabrics and films may be configured as composites, with the material varying from layer to layer in multilayer configurations and also within each layer in both a single layer and multilayer configuration. The layers in multilayer arrangements may be attached to one another by conventional techniques including, but not limited to, stitching, adhesive bonding, heat (or other energy) bonding, and combination thereof. The multilayer configurations are useful in achieving some of the anatomically-specific designs contemplated by certain embodiments of the invention as well as more generally in distributing forces about a relatively wide surface area of the stomach. However, it is understood that certain single layer configurations may also be useful in achieving anatomically-specific designs and in distributing forces.

Further elaborating on the suitable dimensions and configurations for devices placed about the stomach according to certain embodiments of the invention, the shape of the device body may substantially or partially mimic the gross anatomical shape of the entire stomach. Devices according to certain embodiments of the invention may have regions that mimic the shape of certain regions of the stomach, such as, but not limited to, the esophageal junction, the fundus, the pylorus, the inner curvature, the outer curvature, blood vessels in the area of the stomach, and combinations thereof.

Further elaborating on the suitable dimensions and configurations for devices placed about the stomach according to certain embodiments of the invention, the device body may be fabricated such that a user can alter it during placement about the stomach. For example, the device body may have areas amenable to cutout or removal to allow the device to be placed in a way that reduced the compression or dissection of blood vessels. Such removable areas may be fabricated by placing lines of weakness, serrations, and the like in regions of the device likely to encounter specific anatomical features, including blood vessels.

Devices according to certain embodiments of the invention can be configured to achieve anatomically-specific shapes and to distribute forces through the use of areas on the device body amenable to manufacturing techniques such as gathering or bunching, pleating, tucking, cutting, and the like. Such manufacturing techniques may be done prior to delivery to the user or during the method of placing the device. Similarly, features such as gathers, pleats, tucks, gussets, darts, turns and the like may be present in the device body prior to delivery to the user or during the method of placing the device.

According to certain embodiments of the invention, the device body can be relatively rigid rather than flexible and/or stretchable. In such embodiments, the device body is less flexible than the stomach but still flexible enough to be bent around some or all of the stomach and/or still flexible enough to be configured differently from its equilibrium configuration. Some example of types of materials suitable for making a relatively rigid device body include, but are not limited to, the materials described herein for making a relatively flexible device body with the understanding that the device configuration and manufacturing processes may dictate the relative rigidities of device bodies made from such materials. Additionally, reinforced materials, such as plastics strengthened with filler materials like carbon or glass, may be used.

The dimensions and configurations discussed in the prior paragraphs may be used individually or in combination to achieve the relatively smooth contact points where device 100 interacts with stomach 10 and to achieve the relatively wide distribution of constrictive forces about stomach 10.

Devices placed about the stomach according to certain embodiments of the invention are made with laces having suitable dimensions and configurations. In certain embodiments of the invention, the laces are made from a flexible material. In certain embodiments of the invention, the laces are made from a flexible and stretchable material. Some example of types of materials suitable for making laces include elongated structures such as filaments, threads, wires, braids, tubing, strips, and the like made according to conventional techniques; such elongated structures can be made from biocompatible materials such as silicone, polyester, polyvinyl chloride, polyethylene, poly ether-ether ketone, polycarbonate, polyurethane, polypropylene, and nylon

The laces according to certain embodiments of the invention can range in thickness from about 0.05 mm to about 5 mm. To obtain such thicknesses, the lace may be fabricated from a single elongated structure or from multiple elongated structures. The laces may be configured as composites, with the material varying among elongated structures in a multiple elongated structure and also within each elongated structure in both a single elongated structure and a multiple elongated structure configuration. The multiple elongated structures may be attached to one another coaxially, longitudinally, or combinations thereof. The multiple elongated structures may be rolled, braided, wrapped, or otherwise fastened, attached, or secured to one another using conventional techniques.

Further elaborating on the suitable dimensions and configurations for devices placed about the stomach according to certain embodiments of the invention, the possible cross-sectional shape of the laces includes a wide variety of shapes such as, but not limited to, circular, semi-circular, elliptical, semi-elliptical, ovoid, hour-glass, polygonal approximations of such shapes, partial versions of such shapes, asymmetric version of such shapes, and combinations thereof. According the certain embodiments of the invention, the width of the laces may be relatively constant along the length of the lace or it may vary. The variation in the width of the laces can be gradual or abrupt, linear or non-linear. Similarly, according to certain embodiments of the invention, the length of each lace may vary. Suitable cross-sectional shapes, widths, and lengths for the laces are those that allow for the laces to be threaded among the blood vessels in the area of the stomach and through dissections in the omentum while achieving anatomically-specific fit of the device and distributing forces across the stomach.

Devices placed about the stomach according to certain embodiments of the invention are made with suitable means for attaching, fastening, and/or securing the laces to the device body and/or to other laces and for attaching, fastening, and/or securing one device body to another. For example, a device body may be provided with fastening, attachment, and securing points and such coupling points may also be provided on the laces. It is understood that a given coupling point may be used to fasten, attach, or secure one part to another and that the configuration of the coupling point does not necessarily define whether the part is fastened, attached, or secured but rather the nature of the relative motion of one part with another once the parts have been so coupled.

Some examples of suitable coupling points include but are not limited to holes, indents, grommets, loops, hooks, rings, bands, cuffs, snaps, ties, knots, needles, pins, threaded bolts or screws, threaded nuts, clips, crimps, rivets, posts, staples and the like. In certain embodiments of the invention, such as a hole, grommet, loop or similar coupling point, such a point can accommodate a lace passing through it and it is not necessary to have a corresponding mate for the point on the lace. In certain embodiments of the invention, such as hook and loop, snaps, bolts and nuts, or similar paired coupling point, it is necessary that both parts being coupled have a complementary coupling point. For example, a lace may have a male part of a snap near one end of the lace to be attached to a corresponding female part of a snap near an edge of a device body. In certain embodiments of the invention, the coupling points are varied on the device such that a single device (or a single device body or a single lace) may have different types of coupling points. In certain embodiments of the invention, parts may be attached by stitching (including suturing), bonding (including adhesive bonding, energy bonding, and combination thereof), snap joints, one way zip ties, hook and loop, and combinations thereof. Similarly, in certain embodiments of the invention, parts may be secured by stitching (including suturing), bonding (including adhesive bonding, energy bonding, and combination thereof), and combinations thereof. In certain embodiments of the invention, parts may be first fastened using a suitable fastening method and then later secured in place using a suitable securing method.

Another advantage of certain embodiments of the present invention is that the device can conform to the stomach in such a way that the device moves with the stomach. When the device moves with the stomach it can substantially maintain its constrictive function even when the stomach moves. The conforming and adjustable nature of certain embodiments of the invention allows the device to move with the stomach. Optionally, the device may also be anchored to the stomach or abdominal wall at one or more locations around the stomach using conventional anchoring techniques.

According to certain embodiments of the invention, devices may be dimensioned and configured to be inserted using minimally invasive surgical techniques known in the art. Devices may be inserted laparoscopically, transorally, or through other means or procedures for accessing the extra-gastric abdominal space.

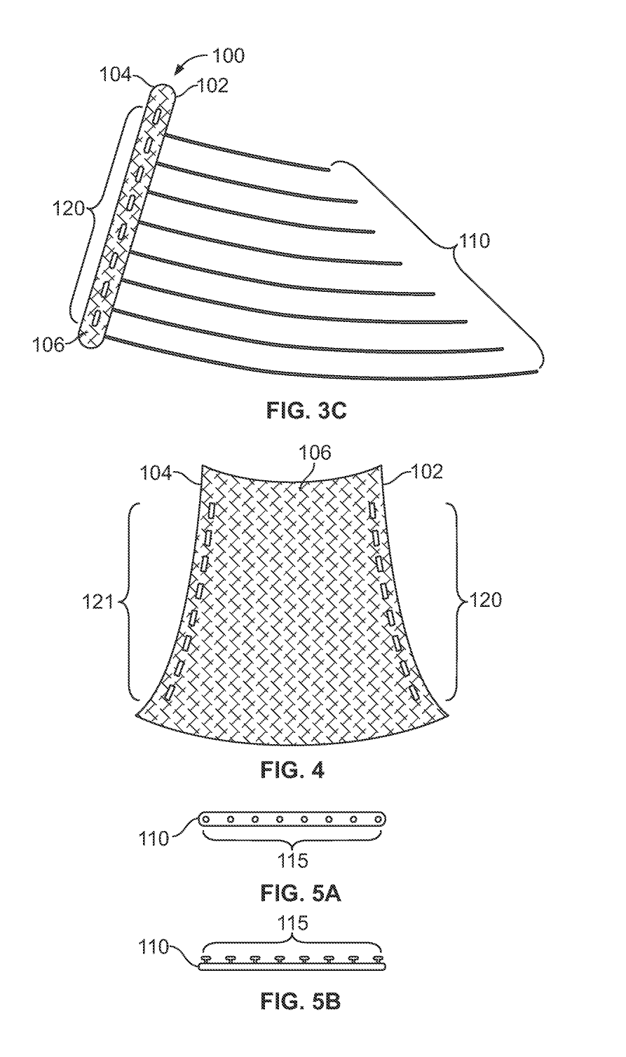

FIGS. 3A, 3B and 3C are each illustrations of different embodiments of the device according to certain embodiments of the invention. It is understood that these depictions of different embodiments of the device are exemplary and non-limiting and that certain features present in a given depiction may optionally be present in other depictions or in embodiments of the device not depicted herein.

Referring now to FIG. 3A, device 100 includes a body 106 that has opposed edges 102 and 104. FIG. 3A depicts body as having a shape that, when folded longitudinally about a longitudinal midline 105, grossly corresponds the anatomical shape of a stomach. As described in more detail herein, body 106 can have a variety of shapes and sizes that correspond to anatomical features of a patient. FIG. 3A depicts device 100 having laces 110 that are attached near edge 104 of device body 106. FIG. 3A depicts fives laces, but it is understood that there may be more or fewer laces on a given device body. FIG. 3A depicts device 100 having coupling points 120 that are attached near edge 102 of device body. FIG. 3A depicts fives coupling points, but it is understood that there may be more or fewer coupling points on a given device body. Also, it is understood that the number of coupling points on a given device body may not match the number of laces on a given device body.

Referring still to FIG. 3A, device body 106 is placed about the stomach and some or all of laces 110 are threaded around and among the blood vessels in the area of the inner curvature of the stomach. In certain embodiments, following blunt dissection of the omentum device body 106 is wrapped about the anterior and posterior of the stomach. Some or all of laces 110 are then threaded through coupling points 120. A given lace may be threaded through the coupling point directly opposed to it or through another coupling point. A given lace may be threaded through more than one coupling point. Some or all of laces 110 are independently and selectively pulled to tighten device 100 about the stomach and optionally to promote the formation of a plication in the stomach. Once the desired amount of constriction is achieved, some or all of the laces 110 are secured such that device body 106 substantially maintains the desired amount of constriction.

Referring now to FIG. 3B, device 100 includes a device body 106 that has opposed edges 102 and 104. FIG. 3B depicts body as having a shape partially mimics the general shape of the stomach to the extent that device body 106 tapers from a narrower width to a wider width. FIG. 3B depicts device 100 having laces 110 that are attached near edge 104 of device body 106 and laces 111 that are attached near edge 102 of device body 106. FIG. 3B depicts eight laces 110 and eight laces 111, but it is understood that there may be more or fewer laces on a given device body or on a given device edge. FIG. 3B depicts device 100 having coupling points 120 that are attached near edge 102 of device body 106 and coupling points 121 that are attached near edge 104 of device body 106. FIG. 3B depicts eight coupling points 120 and eight coupling points 121, but it is understood that there may be more or fewer coupling points on a given device body or on a given device edge.

Referring still to FIG. 3B, device body 106 is placed about the stomach and some or all of laces 110 are threaded around and among the blood vessels in the area of the inner curvature of the stomach. Some or all of laces 111 are threaded around and among the blood vessels in the area of the outer curvature of the stomach and/or through one or more dissections in the omentum. In certain embodiments of the invention, some or all of laces 110 are fastened to some or all laces 111. A given lace may be fastened to more than one other lace. Some or all of laces 110 and 111 are independently and selectively pulled to tighten device 100 about the stomach and optionally to promote the formation of a plication in the stomach. Once the desired amount of constriction is achieved, some or all of the laces 110 are secured such that device body 106 substantially maintains the desired amount of constriction. The laces may be secured using some or all or coupling points 120 and 121.

Referring still to FIG. 3B, in certain embodiments of the invention some or all of laces 110 are threaded around and among the blood vessels in the area of the outer curvature of the stomach and/or through one or more dissections in the omentum after being threaded around and among the blood vessels in the area of the inner curvature of the stomach. Similarly, in certain embodiments of the invention, some or all of laces 111 are threaded around and among the blood vessels in the area of the inner curvature of the stomach after being threaded around and among the blood vessels in the area of the outer curvature of the stomach and/or through one or more dissections in the omentum. Some or all of laces 110 and/or 111 are also threaded through coupling points 120 and or 121. A given lace may be threaded through the coupling point directly opposed to it or through another coupling point. A given lace may be threaded through more than one coupling point. According to certain embodiments of the invention, some or all of laces 110 and 111 are then independently and selectively tightened after having been threaded around the posterior of the stomach and through one or more of coupling points 120 and 121. Tightening some or all of laces 110 and 111 constricts device 100 about the stomach and optionally promotes the formation of a plication in the stomach. Once the desired amount of constriction is achieved, some or all of the laces 110 and 111 are secured such that device body 106 substantially maintains the desired amount of constriction.

Referring now to FIG. 3C, device 100 includes a body 106 that has opposed edges 102 and 104 and laces 110 that are attached near edge 102 of device body 106. FIG. 3C depicts body as having a shape that is relatively narrow as compared to the lateral dimensions of the stomach and relatively short as compared to the length of the laces. In embodiments of the invention such as this one, device body 106 occupies relatively less surface area of the external stomach surface than laces 110 when device 100 is in its constricting state. FIG. 3C depicts device 100 having coupling points 120 that are attached near edges 102 of device body. It is understood that there may be more or fewer coupling points on a given device body or on a given device edge.

Referring still to FIG. 3C, according to certain embodiments of the invention device body 106 is placed about the stomach and some or all of laces 110 are threaded around and among the blood vessels in the area of the inner curvature of the stomach and around and among the blood vessels in the area of the outer curvature of the stomach and/or through one or more dissections in the omentum. Some or all of laces 110 are then threaded through coupling points 120 and/or fastened to some or all of laces 110. Some or all of laces 110 are independently and selectively pulled to tighten device 100 about the stomach and optionally to promote the formation of a plication in the stomach. Once the desired amount of constriction is achieved, some or all of the laces 110 are secured such that device body 106 substantially maintains the desired amount of constriction.

FIG. 4 is an illustration of device body 106 according to certain embodiments of the invention. Device body 106 has opposed edges 102 and 104. FIG. 4 depicts coupling points 120 attached near edge 102 and coupling points 121 attached near edge 104. It is understood that there may be more or fewer coupling points on a given device body. Further, according to certain embodiments of the invention, coupling points may be positioned at places on the device body other than at or near the edge of the device body. The placement of coupling points on a device body can be chosen to, in combination with the dimensions and configuration of the device body and the dimensions and configuration of the laces, to achieve an anatomically-specific fit of the device and to distribute forces across the stomach.

FIG. 5A is a plan view illustration of lace 110 according to certain embodiments of the invention. FIG. 5B is a side view illustration of lace 110 according to certain embodiments of the invention. Lace 110 has couplers 115 arranged along it length. It is understood that there may be more or fewer coupling points on a given device body or on a given device edge. According to certain embodiments of the invention, couplers 115 may be used to fasten, attach, and/or secure lace 110 to device body 106 of FIG. 4 at coupling points 120 and/or 121. FIGS. 5A and 5B depict couplers 115 being uniformly distributed along the length of lace 110, but it is understood that couplers 115 may be distributed in other ways along the length and width of lace 110. Further, FIGS. 5A and 5B depict couplers 115 as being all the same type of coupler, but it is understood that couplers 115 need not be all the same and can be different. In certain embodiments of the invention, lace 110 has both couplers and coupling points 120. In certain embodiments of the invention, device body 106 of FIG. 4 has both coupling points 120 and 121 and couplers 105.

Referring now to FIGS. 4, 5A, and 5B, according to certain embodiments of the invention device body 106 is placed about the stomach and some or all of laces 110 are threaded around and among the blood vessels in the area of the inner curvature of the stomach. In certain embodiments, following blunt dissection of the omentum device body 106 is wrapped about the anterior and posterior of the stomach. In certain embodiments of the invention, some or all of laces 110 are threaded around and among the blood vessels in the area of the inner curvature of the stomach and around and among the blood vessels in the area of the outer curvature of the stomach and/or through one or more dissections in the omentum. In certain embodiments of the invention, some or all of laces 110 are fastened to coupling points 120 and/or 121 and/or fastened to some or all of laces 110. Some or all of laces 110 are independently and selectively pulled to tighten device 100 about the stomach and optionally to promote the formation of a plication in the stomach. Once the desired amount of constriction is achieved, some or all of the laces 110 are secured such that device body 106 substantially maintains the desired amount of constriction. Laces 110 can be secured using coupling points 120 and 121 and couplers 115 or through other means.

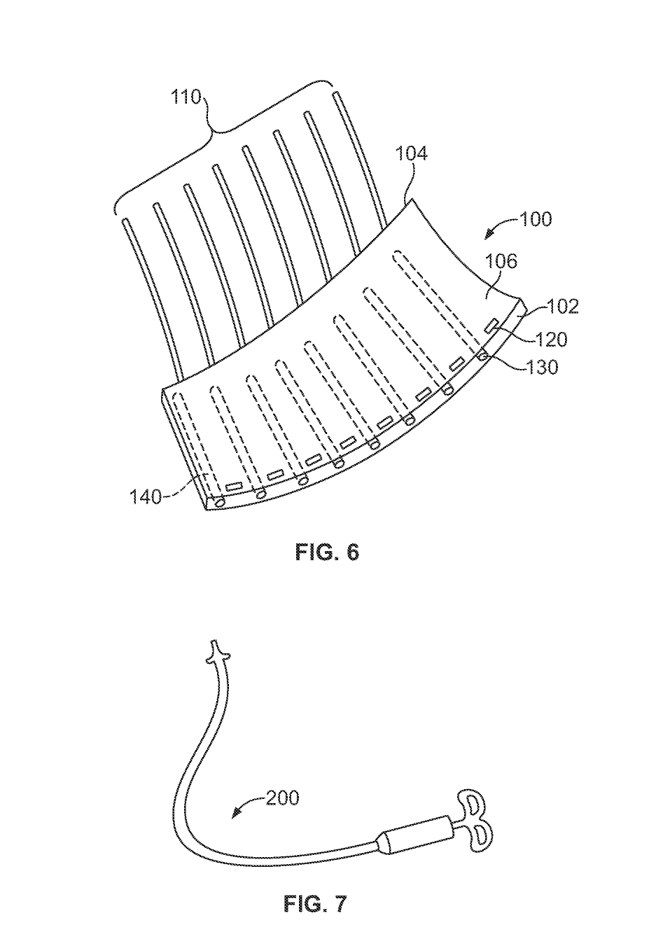

FIG. 6 is a perspective illustration of device 100 according to certain embodiments of the invention in which device 100 includes selectively inflatable bladders 140. FIG. 6 depicts device 100 including body 106 that has opposed edges 102 and 104. FIG. 6 depicts device 100 having laces 110 that are attached near edge 104 of device body 106. FIG. 6 depicts device 100 having coupling points 120 that are attached near edge 102 of device body 106.

FIG. 6 depicts inflatable bladders 140 within device body 106. FIG. 6 depicts eight inflatable bladders 140 but it is understood that a given device body may have more or fewer inflatable bladders. Further, FIG. 6 depicts generally linear inflatable bladders 140 running roughly transverse to the longitudinal axis of device body 106 but it is understood that the inflatable bladders may be any shape and may be arranged other ways within device body 106. Further, inflatable bladders 140 may be on the surface of device body 106 instead of, or in addition to, being within device body 106.

Still referring to FIG. 6, device body 106 includes inflation ports 130 at edge 102. Inflation ports 130 are in fluid communication with inflatable bladders 140. FIG. 6 depicts inflation ports 130 at edge 102 but it is understood that inflation ports 130 may be located at other places on device body 106, such as the exterior surface of device body 106, provided that they are in fluid communication with inflatable bladders 140. Further, FIG. 6 depicts inflatable bladders 140 as each having and being in fluid communication with a single inflation port 130, but it is understood that multiple inflatable bladders 140 may be in fluid communication with a single inflation port 130 such that multiple inflatable bladders 140 may be inflated via a single inflation port. In such embodiments where a single inflation port is in fluid communication with multiple inflatable bladders, the device may optionally include valves that allow for the selective and independent inflation of the inflatable bladders.

According to certain embodiments of the invention, laces 110 may also contain inflatable bladders that allow them to be selectively and independently inflated using one or more inflation ports. The selective and independent inflation of inflatable bladders in or on the device body and in or on the laces is another means of achieving anatomically-specific designs and in distributing forces.

Referring still to FIG. 6, according to certain embodiments of the invention device body 106 is placed about the stomach and some or all of laces 110 are threaded around and among the blood vessels in the area of the inner curvature of the stomach. In certain embodiments, following blunt dissection of the omentum device body 106 is wrapped about the anterior and posterior of the stomach. In certain embodiments of the invention, some or all of laces 110 are threaded around and among the blood vessels in the area of the inner curvature of the stomach and around and among the blood vessels in the area of the outer curvature of the stomach and/or through one or more dissections in the omentum. In certain embodiments of the invention, some or all of laces 110 are fastened to coupling points 120 and/or fastened to some or all of laces 110. Some or all of laces 110 are independently and selectively pulled to tighten device 100 about the stomach and optionally to promote the formation of a plication in the stomach. In certain embodiments of the invention, inflatable bladders 140 are inflated. Once the desired amount of constriction is achieved, some or all of the laces 110 are secured such that device body 106 substantially maintains the desired amount of constriction.

Inflation bladders can be formed using compliant, semi-compliant, and/or noncompliant materials according to conventional techniques. Examples of compliant materials suitable for use in an inflatable bladder as described herein include, but are not limited to: silicone, latex rubber, and polyurethane. Examples of semi-compliant materials include, but are not limited to: nylon, polyethylene, polyester, polyamide and polyurethane. Polyurethane, nylon, polyethylene and polyester can be compliant or semi-compliant materials, depending upon the specific formulation and hardness or durometer of the material as produced. Examples of noncompliant materials that can be used in the construction of inflatable members described herein include, but are not limited to: polyethylene terephalate (PET) and urethane. Urethane can be a compliant, semi-compliant or non-compliant material depending upon its specific formulation and hardness or durometer. Compliant, semi-compliant and noncompliant categories are not solely material limited, but are better defined by their expansion characteristics. Some materials are best suited for use in one of these categories (e.g., silicone and latex work well to make compliant structures), but other materials can be formulated and/or constructed to provide compliant, semi-compliant or noncompliant properties.

FIG. 7 is an illustration of inflation device 200 according to certain embodiments of the invention. Inflation device 200 is capable of forming a fluid-tight seal with inflation ports on a given device. Conventional inflation devices are suitable for use with devices of certain embodiments of the invention.

One advantage of embodiments of the invention having inflatable bladders is that the shape and constrictive forces of the device may be adjusted over time and repeatedly using minimally invasive techniques known in the art. This advantage of being able to adjust an implanted device repeatedly over time may also be realized by virtue of the laces of a device. The laces may be further tightened or loosened using minimally invasive techniques known in the art. For example, laces with multiple couplers along their length may be unsecured, unfastened, moved such that a different coupler becomes coupled to the coupling point on the device body or another lace, and then refastened, tightened, and resecured.

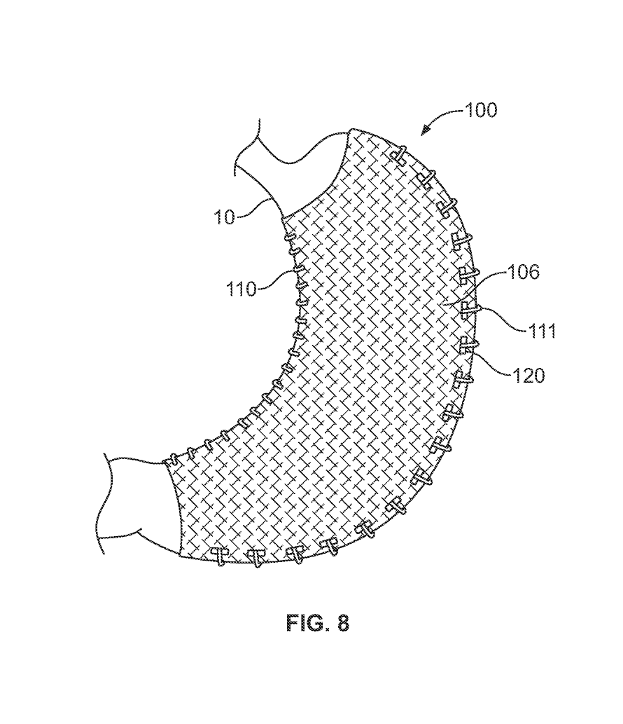

FIG. 8 is an illustration of device 100 according to certain embodiments of the invention in which one device body 106 is placed about the anterior of stomach 10 and another device body (not shown) is placed about the posterior of stomach 10. FIG. 8 depicts the laces 111 of the posterior device body (not shown) fastened through coupling points 110 of device body 106. Similarly, laces 110 of device body 106 are fastened through the coupling points of the posterior device body. FIG. 8 depicts a method according to certain embodiments of the invention where stomach 10 is constrained by two device bodies.

According to certain embodiments of the invention, the device may be provided to the user as at least one device body and at least one lace. According to certain embodiments of the invention, the device may provided to the user as multiple device bodies, some or all of the device bodies having varying dimensions and configurations, and some or all of the laces having varying dimensions and configurations. The device bodies may have dimensions and configurations that vary in features including, but not limited to, shape, size, thickness, flexibility, stretchiness, rigidity and combinations thereof. The laces may have dimensions and configurations that vary in features including, but not limited to, cross-sectional shape, width, thickness, length, flexibility, stretchiness, and combinations thereof.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for the treatment of obesity, the method comprising:
accessing an extra-gastric abdominal space of a patient;
introducing a constricting device into the extra-gastric abdominal space, the constricting device comprising a body with opposed first and second edges and a first plurality of laces with opposed ends;
positioning the body about the stomach of the patient;
threading at least some of the first plurality of laces across the inner curvature of the stomach wherein the laces are threaded so as to avoid compression of blood vessels present near the inner curvature of the stomach;
fastening at least some of the first plurality of laces to the second edge of the body;
tightening at least some of the first plurality of laces to constrict the stomach; and
securing the tightened laces.

2. The method of claim 1 wherein a first end of at least some of the first plurality of laces is attached to the first edge of the body.

3. The method of claim 1 further comprising the step of fastening a first end of at least some of the first plurality of laces to the first edge of the body.

4. The method of claim 1, further comprising creating a fold in the exterior surface of the stomach so that at least two different areas of serosa of the exterior surface of stomach become in contact.

5. The method of claim 4, further comprising maintaining the contact between the at least two different areas of serosa wherein a plication in the stomach membrane is formed.

6. The method of claim 1 wherein the volume of the stomach is reduced as compared to the stomach volume prior to the method taking place.

7. The method of claim 1 wherein the constricting device further comprises a second plurality of laces with opposed ends wherein a first end of each of the second plurality of laces is attached to the second edge of the body.

8. The method of claim 7 wherein at least some of the first plurality of laces are fastened to the second edge of the body by tying at least some of the first plurality of laces to at least some of the second plurality of laces.

9. The method of claim 7 further comprising threading at least some of the second plurality of laces across the inner curvature of the stomach wherein the laces are threaded so as to avoid compression of blood vessels present near the inner curvature of the stomach;
fastening at least some of the second plurality of laces to the first edge of the body;
tightening at least some of the second plurality of laces to constrict the stomach; and
securing the tightened laces.

10. The method of claim 7 further comprising threading at least some of the second plurality of laces through dissections in the omentum.

11. The method of claim 1 further comprising again accessing the extra-gastric abdominal space of a patient;
unsecuring at least one of the first plurality of laces; and
adjusting the amount of constriction the body applies to the stomach.

12. A method for the treatment of obesity, the method comprising:
accessing an extra-gastric abdominal space of a patient;
introducing a constricting device into the extra-gastric abdominal space, the constricting device comprising a first flexible body, a first plurality of laces with opposed ends wherein one end of each of the first plurality of laces is attached to the first flexible body, a second flexible body, and a second plurality of laces with opposed ends wherein one end of each of the plurality of laces is attached to the second flexible body;
positioning the first flexible body and the second flexible body about the stomach of the patient;
threading at least some of the first plurality of laces across the inner curvature of the stomach wherein the laces are threaded so as to avoid compression of blood vessels present near the inner curvature of the stomach;
threading at least some of the second plurality of laces through dissections in the omentum;
fastening at least some of the first plurality of laces to the second flexible body;
fastening at least some of the second plurality of laces to the first flexible body;
tightening at least some of the first or second plurality of laces to constrict the stomach; and
securing the tightened laces.

13. The method of claim 12, further comprising creating a fold in the exterior surface of the stomach so that at least two different areas of serosa of the exterior surface of stomach become in contact.

14. The method of claim 13, further comprising maintaining the contact between the at least two different areas of serosa wherein a plication in the stomach membrane is formed.

15. The method of claim 12 wherein the volume of the stomach is reduced as compared to the stomach volume prior to the method taking place.

16. The method of claim 12 wherein at least some of the first plurality of laces are fastened to the second flexible body by tying at least some of the first plurality of laces to at least some of the second plurality of laces.

17. The method of claim 12 further comprising threading at least some of the second plurality of laces across the inner curvature of the stomach wherein the laces are threaded so as to avoid compression of blood vessels present near the inner curvature of the stomach.

18. The method of claim 12 further comprising threading at least some of the first plurality of laces through dissections in the omentum.

19. A device for the treatment of obesity, the device comprising:
a body having first and second edges wherein the body is configured and dimensioned to be positioned about the stomach of a patient; and
a plurality of laces wherein the laces are configured and dimensioned to be threaded across the inner curvature of the stomach.

20. The device of claim 19 wherein at least some of the plurality of laces are attached to the first edge of the body

21. The device of claim 19 wherein the body comprises a stretchable fabric.

22. The device of claim 19 further comprising a plurality of fastening points near the second edge of the body, wherein the plurality of fastening points are configured to fasten laces.

23. The device of claim 19 further comprising a plurality of laces attached to the second edge of the body.

24. The device of claim 19 further comprising a plurality of fastening points near the first edge of the body, wherein the plurality of fastening points are configured to fasten laces.

25. The device of claim 19 wherein the body further comprises at least one inflatable bladder.

26. The device of claim 25 wherein the body further comprises a plurality of inflatable bladders, the inflatable bladders being selectively and independently inflatable.

27. The device of claim 19 wherein at least some of the plurality of laces comprise inflatable bladders.
